(19) United States
(12) Patent Application Publication
(10) Pub. No.: US 2013/0079607 A1
(43) Pub. Date: Mar. 28, 2013

(54) FIBEROPTIC PROBE FOR MEASURING TISSUE OXYGENATION AND METHOD FOR USING SAME

(71) Applicants: Dan Gareau, Portland, OR (US); Steve Jacques, Portland, OR (US); John Hunter, Portland, OR (US)

(72) Inventors: Dan Gareau, Portland, OR (US); Steve Jacques, Portland, OR (US); John Hunter, Portland, OR (US)

(73) Assignee: OREGON HEALTH & SCIENCE UNIVERSITY, PORTLAND, OR (US)

(21) Appl. No.: 13/575,241

(22) PCT Filed: Jan. 25, 2011
(86) PCT No.: PCT/US11/22467

§ 371 Date: Dec. 10, 2012

Related U.S. Application Data
(60) Provisional application No. 61/298,120, filed on Jan. 25, 2010.

(51) Int. Cl. A61B 5/1455

(52) U.S. Cl. CPC A61B 5/14552
USPC 600/323

(57) ABSTRACT

Embodiments herein relate to the field of medical monitoring, and, more specifically, to a fiberoptic probe for monitoring tissue oxygenation and a method for using such a probe. A non-invasive method of measuring tissue oxygenation includes, in some embodiments, illuminating a tissue surface with a first fiberoptic fiber, receiving light from the tissue surface with a second fiberoptic fiber, measuring the absorption spectra of oxy- and deoxy-hemoglobin in the light, and calculating a tissue oxygenation value based on the absorption spectra.

### CROSS REFERENCE TO RELATED APPLICATIONS

The present application claims priority to U.S. Provisional Patent Application No. 61/298,120, filed Jan. 25, 2010, entitled “Fiberoptic Probe for Monitoring Tissue Perfusion and Method for Using Same,” the entire disclosure of which is hereby incorporated by reference in its entirety.

### GOVERNMENT INTERESTS

This invention was made with Government support under Grant/Contract No. R01-HL084013 awarded by the National Institutes of Health. The Government has certain rights in the invention.

### TECHNICAL FIELD

Embodiments herein relate to the field of medical devices and methods, and, more specifically, to a fiberoptic probe to obtain an optical spectrum, a spectral analysis for measuring/monitoring tissue oxygenation, and a method for using such a probe.

### BACKGROUND

Oxygen saturation and blood volume fraction are critical indicators of tissue viability. However, current methods of noninvasive monitoring are insufficient in that they require the presence of a strong pulse and consequently are not effective for measuring oxygen saturation and blood volume fraction in tissue with a weak pulse or in bulk tissue.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1A shows a sample experimental spectrum in accordance with embodiments 0 herein. Also shown for reference are the reflectance spectra measured and predicted at the same blood content (B=0.0039) for purely HbO₂ (S=1) and Hb (S=0);

FIG. 1B illustrates a fiberoptic device in accordance with embodiments herein;

FIGS. 2A, 2B, and 2C show various features of a fiberoptic probe, in accordance with various embodiments.

FIG. 3 is a graph illustrating the output of an exemplary implantable device when attached to a pig that was sacrificed by lethal injection, in accordance with various embodiments; and

FIG. 4 is a graph illustrating sample spectra that yielded the saturation measurements shown in FIG. 2, in accordance with various embodiments.

### DETAILED DESCRIPTION OF DISCLOSED EMBODIMENTS

In the following detailed description, reference is made to the accompanying drawings, which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “NB” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

In various embodiments, methods, apparatuses, and systems for spectroscopic monitoring of tissue oxygenation are provided. In exemplary embodiments, a computing device may be endowed with one or more components of the disclosed apparatuses and/or systems and may be employed to perform one or more methods as disclosed herein.

Some embodiments provide a fiberoptic probe that noninvasively measures blood content and hemoglobin saturation by contact from the surface of tissue. This enables rapid noninvasive measurement of vital signs of patients and in tissues with a weak or nonexistent pulse. The technology is therefore superior to existing pulse oxymeters and laser Doppler flowmeters, which require the presence of a strong pulse and consequently are not effective for measuring oxygen saturation and blood volume fraction in tissue with a weak pulse or in bulk tissue.

In certain embodiments, the device may include a probe that includes at least two fiberoptic fibers that terminate at a surface of the probe, generally adjacent to one another. In some embodiments, the first fiberoptic fiber transmits light from a light source to the tissue surface, and the second fiberoptic fiber receives light from the tissue surface and transmits it to a spectrometer. In various embodiments, the first and second fiberoptic fibers are separated from one another by about 2 mm to about 4 mm on the surface of the probe, for instance, about 2.5 mm to about 3.5 mm. In an embodiment, a distance of 3 mm may be utilized between the ends of the fibers, which appears to be a particularly beneficial distance for obtaining measurements in superficial tissue using visible wavelengths. In embodiments, the light used may be in the visible wavelength range, such as 480-700 nm wavelength.

In some embodiments, the device also includes a computing device coupled to the spectrometer, and the computing device is configured to generate a tissue oxygenation value and total blood volume content based on the light transport measured by the spectrometer. The spectrometer may be any commercially available spectrometer, and the computing device, may be, for instance, a laptop, personal computer, or PDA-type device.

In embodiments, the probe may measure the light transport in tissue between the two or more fiberoptic fibers. A spectroscopic analysis may be carried out, in embodiments, that utilizes the absorption spectra of oxy- and deoxy-hemoglobin and optical diffusion theory, incorporating the tissue scattering properties and blood absorption to estimate the blood volume fraction (perfusion) and the oxygen saturation of hemoglobin HbO₂/(Hb+HbO₂) in the mixed arterio-venous vasculature.

In other embodiments, a spectroscopic method of assessing the blood perfusion/oxygenation status of a tissue is provided that uses a simple, two-optical-fiber probe inserted into a subject, for instance via laparoscopy and/or during cosmetic surgery. In some embodiments, the method includes illuminating a tissue surface with a first fiberoptic fiber; receiving light from the tissue surface with a second fiberoptic fiber; measuring the absorption spectra of oxy- and deoxy-hemoglobin in the light; and calculating a tissue oxygenation value based on the absorption spectra. In contrast to currently available techniques for monitoring oxygenation including pulsed oxymetry, and Doppler flowmetry, this steady-state measurement does not rely on vascular flow and may therefore measure oxygenation in the blood of bulk tissue (for instance, in capillaries). In conventional technologies that use deep probing (for instance, about 8 cm), it is necessary to use the infrared wavelength for sampling. By contrast, the present methods make use of shallow (<5 mm) monitoring of tissue oxygenation.

In an embodiment, which is referred to herein as the alpha device, the probe may provide for light emission from an end or distal tip of the device. As described below in greater detail, if the probe/light is facing the tissue incorrectly, there may be a reduction in received data quality. In certain situations, for example due to particular surgical approaches, it may be difficult to orient the probe in the proper angle as part of surgery. Thus, in some embodiments, it may be easier to insert a wire down a tube or conduit and align the side of the wire to face the tissue being monitored. This may be done directly (by flexing the fiber) or by using a reflective surface to redirect light. Thus, an embodiment provides for light emission from a source to be from the side of the device (the “side-fire” device, also referred to herein as the beta device).

Using an esophagectomy as an illustrative example, in order to mobilize the stomach tissue that will become the conduit from the arteries that tether it, the short gastric and left gastric arteries may be surgically transected. Thus the right gastroepiploic artery is the sole remaining vessel supplying the gastric conduit and, consequently, blood supply is decreased to the very tissue that must be anastamosed to the remaining esophagus in the subject's neck. Unfortunately, in up to 20% of the cases the anastamosis fails, requiring surgical intervention to fix leakage at the anastomosis connecting the gastric conduit to the pharynx. Many factors influence the outcome, but adequate oxygenation at the anastamosis is important to success of the surgery.

There is currently no commercial means to monitor the status of the anastomosis, and failures, in the form of leaks, present too late for preventative effective intervention. Anastomotic leak contributes substantially to the 5% mortality rate associated with esophagectomy, therefore any method of early detection for the scheduling of pre-failure intervention may improve patient outcome. Detection of a significant decrease in normal blood oxygenation at the anastomosis may alert the surgeon that the conduit or anastamosis may be at risk for ischemic injury, and further diagnostic and therapeutic intervention may be scheduled. Thus, the probe system disclosed herein moves steady-state optical spectroscopy into clinical practice. The saturation measured by the alpha design, if deemed to be dangerously low at the conclusion of the esophagectomy surgery, may warrant the attachment of the beta design to be left in place during the days following surgery to monitor recovery from ischemia or identify non-recovery to schedule surgical intervention prior to the predicted anastomosis failure.

Embodiments herein may be used to measure/monitor oxygenation in a variety of situations, including anastomosis, vascular surgery (such as monitoring the effected distal region), cosmetic surgery (such as monitoring a repositioned tissue flap), etc.

As disclosed herein, fiberoptic spectroscopy may be implemented with a small footprint, for instance, using two 1 mm diameter optical fibers placed a short distance apart, such as from about 2 mm to about 4 mm apart, for instance about 2.5 mm, 3 mm, or 3.5 mm apart. This may help avoid the dangers associated with placing electrical components inside the subject. The probe may measure steady-state light signals, as opposed to a pulse-oxymetry unit, which must lock onto a weak pulsatile signal in order to extract information. Moreover, the probe may be less sensitive to the pO₂ of the arterial blood being delivered to a tissue, and more sensitive to the oxygen extraction by the tissue. Hence, if arterial blood flow is inadequate, despite being well oxygenated, the mixed arterio-venous oxygen saturation may drop because O₂ extraction outpaces O₂ delivery.

The oxyhemoglobin (HbO₂) and deoxyhemoglobin (Hb) molecules exhibit distinct absorption properties in the spectral range centered between 550 and 600 nm, which contains the alpha and beta absorption bands. The spectroscopic analysis may utilize the absorption spectra of oxy- and deoxy-hemoglobin and optical diffusion theory, in some embodiments, incorporating the tissue-scattering properties and blood absorption to estimate the blood volume fraction and the oxygen saturation of hemoglobin HbO₂/(Hb+HbO₂) in the mixed arterio-venous vasculature.

In one specific example, alpha probe devices were created using standard machining and fiber polishing tools. The clear, 8-mm diameter cylindrical probe tip had 1-mm-diameter holes drilled parallel to its axis at a separation distance of about 3 mm. The delivery fiber and a second identical fiber for light collection were polished along with the probe tip face to achieve one clear planar surface. Because the probes were hand made, the separation distance between the fibers varied from about 2.5 to about 3.5 mm. Each probe was cataloged by noting the radial fiber separation and calibrated by a measurement on a reflectance standard consisting of an epoxy resin block with titanium dioxide as scatterer. The optical properties of the standard (at 532 nm) were: μₛ′=21 cm⁻¹, mua=0, x cm⁻¹, g=0.7. Probes were then sterilized and hermetically sealed (Sterrad, ASP, Irvine Calif.). In the operating room, the two sterile 4 meter-long fibers delivered and collected light between the surgeons and the “scrubbed in” engineer outside of the surgical sterile zone.

The probe was introduced percutaneously into the abdominal cavity through a 10-mm-diameter trocar, and placed on the gastro-esophageal anastamosis by the surgeons. Spectra were collected before and after division of the short gastric arteries and after division of the left gastric arteries. At each time point, five measurements were taken in rapid succession at each of three locations within 2 cm of a marking stitch, which identified the measurement location on the caudal side of the anastamosis during creation of the gastric conduit. The integration time for each measurement was about 200 ms, but could be adjusted to obtain a reliable measurement. Each spectrum was recorded with its integration time, and subsequent data analysis used the counts per spectral bin divided by the integration time, [counts/bin/s].

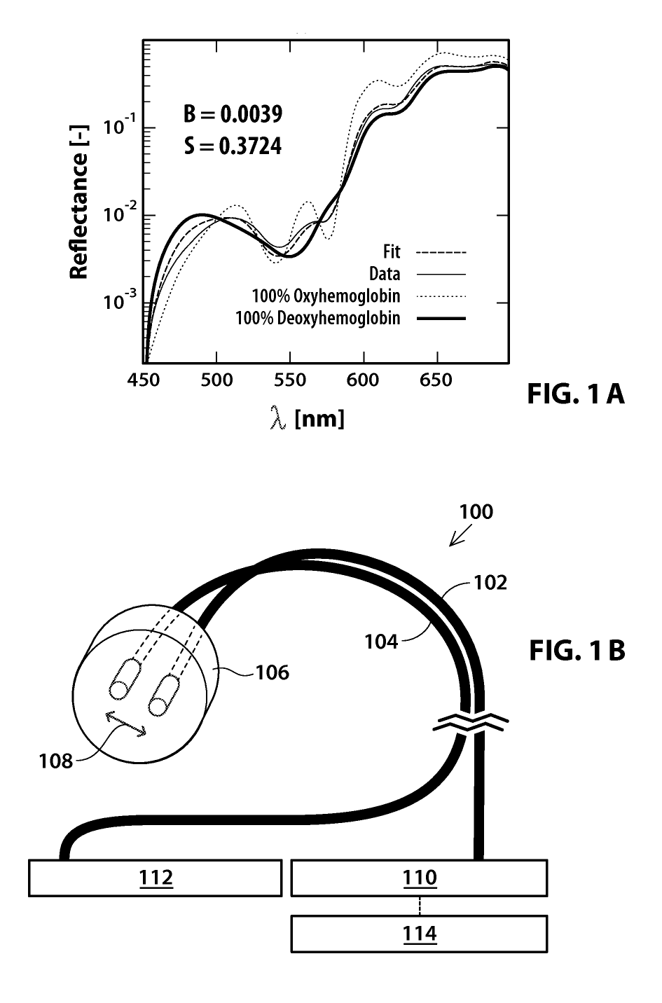

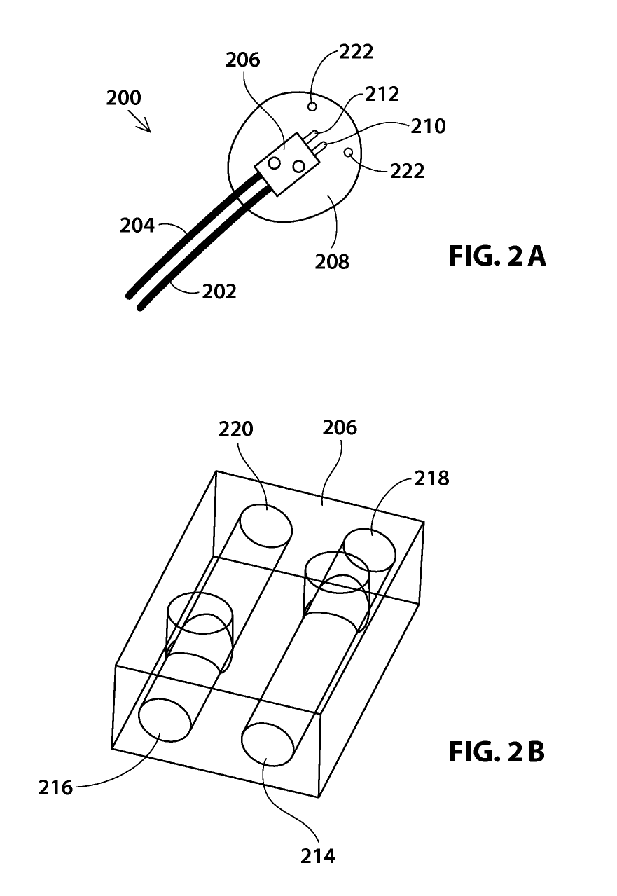

FIG. 1A shows a sample spectrum specified by the fitting parameters: blood volume content (B) and oxygen saturation (S=HbO₂/(Hb+HbO₂)). The “Fit” curve shows the predicted reflectance spectrum for the blood content and saturation. Also shown for reference are the reflectance spectra predicted at the same blood content (B=0.0039) for purely HbO₂ (S=1) and Hb (S=0).

FIG. 1B illustrates a fiberoptic device 100 in accordance with embodiments herein. Device 100 has a first fiberoptic fiber 102 and a second fiberoptic fiber 104 terminating in housing 106. The distal tips/ends of fibers 102, 104 terminate at a surface of housing 106 and are separated by a distance 108, such as about 3 mm. Fiber 102 is coupled to a light source 110, and fiber 104 is coupled to a spectrometer 112. The spectrometer 112 may further comprise, or be coupled to, a computing device 114 to control spectrometer 112 and/or to process certain calculations, analyses, store data, etc.

In a specific embodiment, the probe housing held two fiber faces (one for illumination and one for collection) to the tissue surface so that the fibers were at a 90-degree angle to the tissue. Because glass is generally not safe to insert into patients, plastic fibers (NT02-534, Edmund Optics, Barrington, N.J.) were used, for example a 1 mm core diameter fiber. A white light source (L-2000-LL, Ocean Optics, Dunedin, Fla.) was coupled to the plastic fiber with a standard SMA connector (11040A, Thor Labs, Newton, N.J.). A thin glass fiber of 100 μm core diameter (BFL22-200, Thor Labs, Newton, N.J.) was coupled between the collection fiber and the spectrometer (QE 65000, Ocean Optics, Dunedin, Fla.), which improved the spectral resolution of the spectrometer. The spectrometer was controlled by a laptop computer (Dell Computer, Round Rock, Tex.) running the Windows XP Professional operating system.

In an exemplary embodiment, a fiberoptic device comprises a probe comprising at least a first fiberoptic fiber and a second fiberoptic fiber, wherein the first and second fiberoptic fibers terminate at or near a surface of the probe; a visible wavelength light source coupled to the first fiberoptic fiber; and a spectrometer coupled to the second fiberoptic fiber and configured to measure light transport in tissue adjacent to the surface of the probe.

In an alternative embodiment, the detected light fiber (fiber 104 in FIG. 1) may be coupled to a fiber bundle with multiple-around-one, such as 6-around-one, circular fibers on the end connecting it to the spectrometer, such as in a linear array.

Monte Carlo models indicate that, in certain embodiments, for the 3 mm radial fiber separation between irradiance and remittance, the light traveled about 1 cm through the tissue. The diffuse reflectance spectrum recorded by the spectrometer carried information about blood content and saturation. At each wavelength, the scattering was specified by a polynomial fit of three parameters. These parameters were allowed to vary along with the saturation and blood fraction for a total of 5 fitting variables to predict the reflectance spectrum which was fit with a least squares regression algorithm (Nelder-Mead unconstrained nonlinear minimization). The scattering and absorption lead to the predicted diffuse reflectance at the known radial separation distance of the fiber tips in contact with the tissue at each wavelength. The predicted spectrum was fit to the measured spectrum, specifying the saturation and blood volume fraction.

The total absorption by the tissue was calculated as a linear superposition of the absorption due to the chromophores oxygenated (μ_{aOxy}) and deoxygenated hemoglobin (μ_{aDeoxy}).

μ_{aTissue}=B(Sμ_{aOxy}+(1−S)μ_{aDeoxy})+Wμ_{aWater} (1)

In equation 1, B is the fraction of blood in the tissue, S is the oxygen saturation fraction and W is the fraction of water in the tissue, which was assumed to be 0.75. The absorption coefficient of the tissue (μ_{aTissue}) was specified for each wavelength with the fitting parameters B and S and computing equation 1. Equation 2 specifies the scattering coefficient (μ′_{sTissue}) with the fitting parameter a and the fractions of scattering expected to be Rayleigh scattering (f_{Rayleigh}=0.63) and Mie scattering (f_{Mie}=0.37).

$${μ}_{sTissue}^{′} = a ⌊ {{f}_{Rayleigh} ( \frac{λ}{500 nm} )}^{- 4} + {{f}_{Mie} ( \frac{λ}{500 nm} )}^{- 1} ⌋ \qquad ( 2 )$$

The diffuse reflectance was calculated from μ_{aTissue} using the scattering (specified by equation 2), the radial fiber separation (ρ) as catalogued, the refractive index of the tissue (assumed to be n=1.4). The calculated reflectance was subtracted from the measured data, yielding an error that was minimized by iterating the guesses of the fitting parameters until the blood factors B and S and the scattering parameters a and b were converged upon (see equation 3):

$$R ( ρ ) = b \frac{1}{4 π {μ}_{t}^{′}} [ ( {μ}_{eff} + \frac{1}{{r}_{1}} ) \frac{exp ( - {μ}_{eff} {r}_{1} )}{{r}_{1}^{2}} + ( \frac{3}{4} A + 1 ) ( {μ}_{eff} + \frac{1}{{r}_{2}} ) \frac{exp ( - {μ}_{eff} {r}_{2} )}{{r}_{2}^{2}} ] \\ {r}_{1} = \sqrt{{z}_{0}^{2} + {ρ}^{2}} \\ {r}_{2} = \sqrt{{( {z}_{0} + 2 {z}_{b} )}^{2} + {ρ}^{2}} \qquad ( 3 )$$

where μₜ′=μ_{aTissue}+μ′_{sTissue}, μ_{eff} is the effective attenuation coefficient or reciprocal of diffusion length, A is a specular reflection factor given by A=(1+rᵢ)/(1−rᵢ), rᵢ=0.6681+0.0636n+0.7099/n−1.4399/n², n is the tissue refractive index, z₀=1/μₜ′ and z_{b}=2AD, where D=1/3μₜ′, and b is the final fitting parameter.

Of 23 esophagectomy subjects studied, not all were measured at all three major time-points due to surgical circumstances. The mean saturation and blood volume fraction were computed and a paired, 1-tailed student T-test was performed to show the decrease in saturation with arterial ligation. The mean and standard deviation for the baseline oxygen saturation were S=0.48+/−0.24 and after ligation of the short gastric arteries were S=0.40+/−0.19, based on n=11 patients. The difference in measurements had a significance of p=0.111. The oxygen saturation decreased from the measurement after ligation of the short gastric arteries (S=0.38+/−0.19) to the measurement after ligation of the left gastric artery (S=0.32+/−0.19) based on n=20 patients (p=0.046). The oxygen saturation decreased from the baseline measurement (S=0.47+/−0.23) to the measurement after ligation of the left gastric arteries (S=34+/−0.19) based on n=12 patients (p=0.008). Relative to baseline value, the blood volume fraction increased by 166% after conduit creation (p=0.06) and by 256% following pull-up (p=0.02).

Compared to patients without anastomotic complications, the seven patients who manifested anastomotic complications had greater intraoperative changes in S (50.2% decrease from baseline versus 18.9%, p=0.02). However, the blood volume fraction (160.2% vs. 169.2%, p=0.9) did not differ between patients with and without anastomotic complications. Four patients had ischemic conditioning by short gastric vessel division at a median of 94 days prior to esophagectomy. Compared to patients who underwent immediate reconstruction, those who underwent ischemic conditioning had significant differences in BVF relative to baseline (182.5% versus 73.1%, p=0.02). However, S did not decrease significantly (29.3% decrease from baseline vs. 29.8%, p=0.9) for patients with ischemic conditioning versus those without prior ischemic conditioning after conduit creation.

The alpha device and technique disclosed herein reliably determined the blood saturation and blood volume fraction in the gastric conduit through laparoscopic ports during esophagectomy. The data and fit shown in FIG. 1A is about average for the entire data set in terms of accuracy of the fit. The fit tracks the data reasonably well over the entire spectrum with minor errors around 550 and 475 nm.

The oxygen saturation decreased over the surgery with the division of the arteries that supply blood, particularly the left gastric artery. Of the 23 patients studied, the seven patients that experienced anastomotic complications were shown to have a greater decrease in tissue blood saturation than those who had no complications. Thus, intra-operational hemodynamics are only part of the story, and there are healing dynamics that play out in the recovery days following surgery that also impact the oxygen saturation of the blood in the tissue and influence viability. Such dynamics may be the possible increase of blood supply by the left gastroepiploic artery that remains intact throughout the surgery.

Thus, in an exemplary embodiment, a method of measuring tissue oxygenation comprises illuminating a tissue surface with a first fiberoptic fiber; receiving light from the tissue surface with a second fiberoptic fiber, wherein the light received by the second fiberoptic fiber comprises a visible wavelength range tissue spectrum; measuring the absorption spectra of oxy- and deoxy-hemoglobin in the light; and calculating a tissue oxygenation value based on fitting the tissue spectrum in the visible wavelength range to the absorption spectra of oxy- and deoxy-hemoglobin.

In a further embodiment, a probe that may be sutured to the conduit and remain in place during the post-operative recovery period would enable monitoring of tissue such that non-reperfusing cases can be scheduled for surgical intervention before leaks occur at the anastamosis site. Such a probe that may be sutured into position was designed and tested as the beta device. In some embodiments, the beta device (which works generally the same way as the alpha device, but which may have a different light-emission configuration in some embodiments) may be sutured onto the tissue, for instance an anastomosis or any other type of tissue in which it is desirable to monitor oxygenation, in order to monitor tissue vital signs over long periods of time.

Further, to address concerns pertaining to inflammation and/or fibrosis that may caused by implantation of a probe, the probe may be coated with a biocompatible coating prior to implantation. The coating may be applied by any suitable process such as spray deposition, vapor deposition, dip-coating, etc. For example, the working end of a probe may be dipped into silicone rubber and allowed to dry/cure thus enclosing the probe with an outermost biocompatible coating prior to implantation.

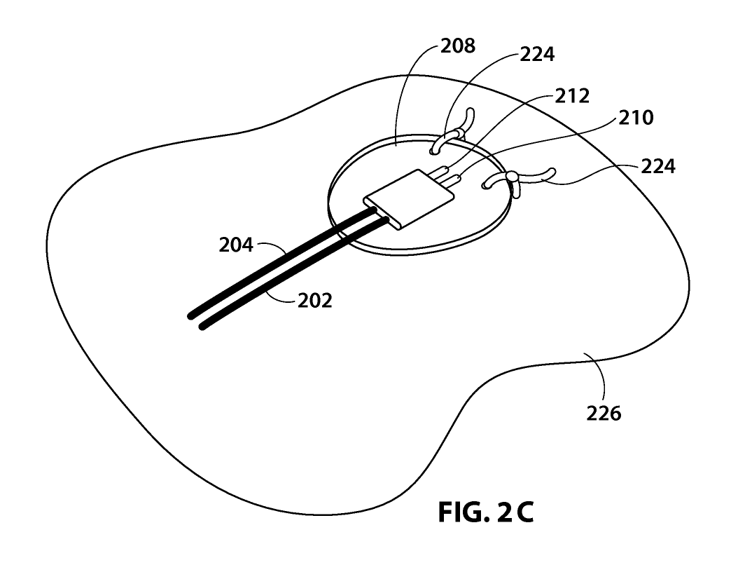

FIGS. 2A, 2B, and 2C show various features of a fiberoptic probe 200, in accordance with various embodiments. Probe 200 includes first and second fiberoptic fibers 202, 204 terminating in housing 206. Housing 206 and fibers 202, 204 are partially disposed within waveguide 208, which may be a UV-cured optical waveguide in an embodiment. To redirect light from or along fibers 202, 204, metal rods 210, 212, such as fabricated from stainless steel, are inserted into the opposite ends of housing 206. Polished or mirrored surfaces, such as at 45° angles, when properly aligned redirect light as desired. In alternative embodiments, mirrors or other reflective surfaces may be used. Alternatively, the fibers may be flexed to provide the desired configuration/alignment.

FIG. 2B illustrates a schematic diagram of housing 206. Ports 214, 216 are provided for insertion of fibers 202, 204 and ports 218, 220 are provided for insertion of rods 210, 212. In this embodiment, fibers 202, 204 do not extend all the way to the housing surface, but rather are effectively extended by the rods (or other such device). Such a configuration can be termed “near a surface of the probe” as the terminal portion of each fiber is effectively at the probe surface.

Probe 200 may be coupled to tissue, such as by sutures 224. Waveguide 208 has a plurality of holes 222 provided to permit sutures to pass therethrough and to secure the waveguide to tissue. FIG. 2C shows probe 200 sutured to exemplary tissue 226 in surgery.

In a specific embodiment, the beta device includes a beveled stainless steel rod, for instance, made from 316 L medical grade stainless steel, a black plastic probe tip housing (for instance, a MacMaster Carr 87875K37), UV-cured optical waveguide (for instance, from Norland Products, NOA 68), a fiberoptic cable (for instance, an Edmund Optics NT02-534), medical grade super glue (for instance, Loctite 4011), and Gortex™ for suturing the device to tissue, for instance gastric conduit.

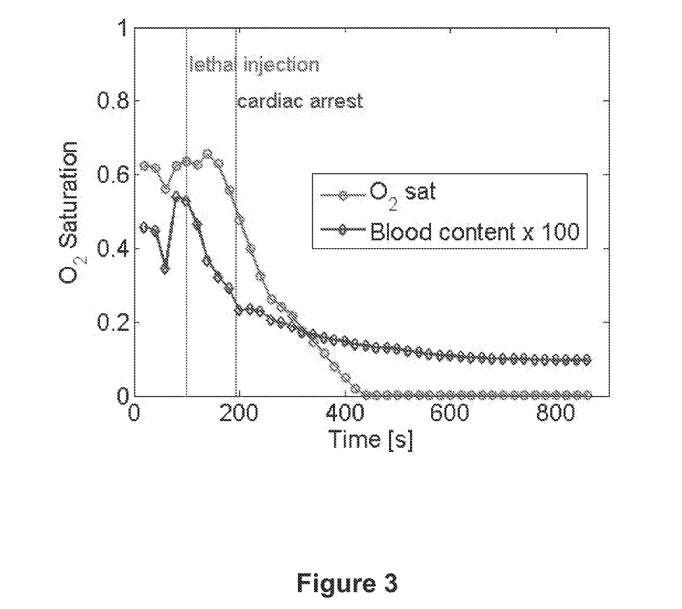

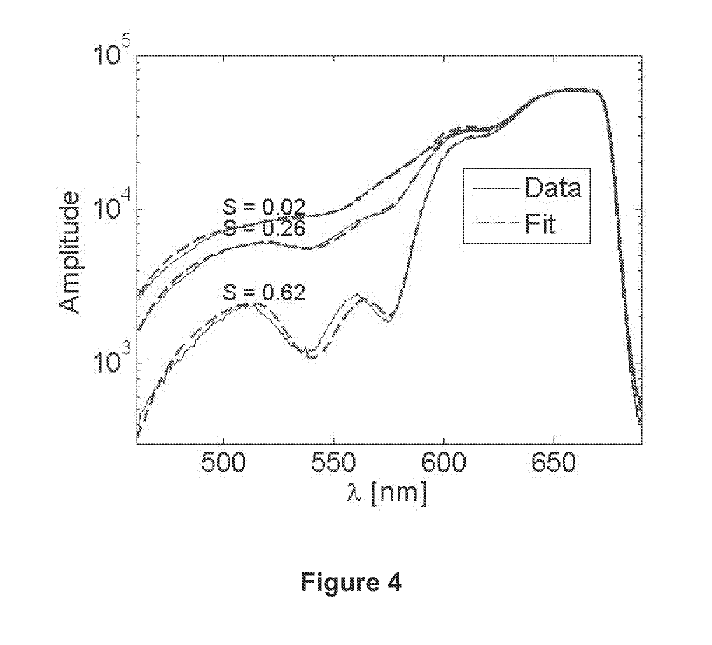

In a specific example, the beta device described above was tested in an animal in an IACUC-approved add-on to a prescheduled animal euthanasia. Before sacrifice, the surgeon attached the device to the stomach tissue by means of two stitches through the laparoscope port with the Hunter grips. FIG. 3 shows the output of the first implantable (end/tip) alpha device. This result accurately (˜±0.02) shows the oxygen supply decrease to zero after vascular shut-down. The overall blood content pooled away from the measurement site on the top surface of the stomach. The stable nature of the probe and measurement were enabled by the focus on the spectroscopic region of the 5 isobestic points and appears to be extremely robust. To illustrate the actual fits to the data, three representative time points were chosen (low, medium, and high saturation S) as shown in FIG. 4.

The spectroscopic approach has been improved in the beta device. The cut-off on the right hand side of FIG. 4 was achieved by passing the light source (Ocean Optics HL 2000-HP) through an optical filter (Semrock FF01-554/211). This sharp edge helped by providing a calibration (location of half maximum) in the fitting algorithm which was much improved over the alpha device testing.

Fiberoptic spectroscopy (FOS) utilizes the differential spectral absorbance characteristics of oxy- and deoxy-hemoglobin to determine oxygen saturation (OSat) and blood volume fraction (BVF) within tissues. In one specific example, FOS was used to measure OSat and BVF in the distal tip of the gastric conduit at baseline, after division of the short gastric vessels, left gastric vessels, gastric tube creation, and conduit pull-up. OSat and BVF readings were normalized to baseline and correlated to clinical outcomes.

Between 2008 and 2009, 23 patients underwent minimally invasive esophagectomy. Four patients had ischemic conditioning by short gastric vessel division at a median of 94 days prior to esophagectomy. Seven patients developed an anastomotic leak or stricture. OSat decreased from 47.5% at baseline to 32.3% after conduit creation (p=0.002) and then to 36.4% after pull-up (p=0.02). Relative to baseline value, BVF increased by 166% after conduit creation (p=0.06) and by 256% following pull-up (p=0.02). Compared to patients without anastomotic complications, those who manifested anastomotic complications had greater intraoperative changes in OSat (18.9% decrease from baseline versus 50.2%, p=0.02). However, BVF (160.2% vs. 169.2%, p=0.9) did not differ between patients with and without anastomotic complications. Compared to patients who underwent immediate reconstruction, those who underwent ischemic conditioning had significant differences in BVF relative to baseline (182.5% versus 73.1%, p=0.02). However, OSat did not decrease significantly (29.3% decrease from baseline vs. 29.8%, p=0.9) for patients with ischemic conditioning versus those without prior ischemic conditioning after conduit creation.

The degree of intraoperative gastric ischemia resulting from gastric conduit creation is associated with the development of anastomotic complications. In patients undergoing ischemic conditioning, decreases in BVF indicate less venous congestion in the gastric conduit. Thus, FOS may be useful in assessing the changes in conduit perfusion/oxygenation during esophagectomy.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A fiberoptic device, comprising:
a probe comprising at least a first fiberoptic fiber and a second fiberoptic fiber, wherein the first and second fiberoptic fibers terminate at or near a surface of the probe;
a visible wavelength light source coupled to the first fiberoptic fiber; and
a spectrometer coupled to the second fiberoptic fiber and configured to measure light transport in tissue adjacent to the surface of the probe.

2. The fiberoptic device of claim 1, further comprising a computing device electrically coupled to the spectrometer, wherein the computing device is configured to generate a tissue oxygenation value and total blood volume content based on the light transport measured by the spectrometer.

3. The fiberoptic device of claim 2, wherein the computing device is configured to generate a tissue oxygenation value using absorption spectra of oxy- and deoxy-hemoglobin and a scattering spectrum of bulk tissue.

4. The fiberoptic device of claim 3, wherein the tissue oxygenation value comprises an estimate of the blood volume fraction and oxygen saturation of hemoglobin HbO₂/(Hb+HbO₂) in mixed arterio-venous vasculature of bulk tissue.

5. The fiberoptic device of claim 1, wherein the surface of the probe is a distal tip surface.

6. The fiberoptic device of claim 1, wherein the surface of the probe is a side surface.

7. The fiberoptic device of claim 1, further comprising a plastic probe tip housing that houses at least part of the first and second fiberoptic fibers.

8. The fiberoptic device of claim 1, wherein the first and second fiberoptic fibers are at least partially disposed in a UV-cured optical waveguide.

9. The fiberoptic device of claim 8, further comprising one or more mirrored surfaces disposed in the UV-cured optical waveguide and configured to redirect light.

10. The fiberoptic device of claim 9, wherein the one or more mirrored surfaces comprise one or more cylindrical metal components having 45° angled mirrored surfaces.

11. The fiberoptic device of claim 1, wherein the first and second fiberoptic fibers are spaced from about 2 mm to about 4 mm apart at the surface of the probe.

12. The fiberoptic device of claim 1, wherein the first and second fiberoptic fibers are spaced from about 2.5 mm to about 3.5 mm apart at the surface of the probe.

13. The fiberoptic device of claim 1, wherein the first and second fiberoptic fibers are spaced about 3 mm apart at the surface of the probe.

14. The fiberoptic device of claim 1, further comprising a cable that encloses at least a portion of the fiberoptic fibers.

15. The fiberoptic device of claim 1, further comprising a suture substrate for securing the probe surface against a tissue.

16. The fiberoptic device of claim 15, wherein the suture substrate comprises a UV-cured optical waveguide.

17. The fiberoptic device of claim 16, wherein the UV-cured waveguide is configured to correspond in shape and/or size to one or more features of a surgical site.

18. The fiberoptic device of claim 1, wherein the spectrometer is coupled to the second fiberoptic fiber by multiple-around-one circular fibers.

19. The fiberoptic device of claim 1, further comprising an outermost biocompatible coating disposed on at least a portion of the probe.

20. The fiberoptic device of claim 19, wherein the biocompatible coating comprises silicone rubber.

21. A method of measuring tissue oxygenation, comprising:
illuminating a tissue surface with a first fiberoptic fiber;
receiving light from the tissue surface with a second fiberoptic fiber, wherein the light received by the second fiberoptic fiber comprises a visible wavelength range tissue spectrum;
measuring the absorption spectra of oxy- and deoxy-hemoglobin in the light; and
calculating a tissue oxygenation value based on fitting the tissue spectrum in the visible wavelength range to the absorption spectra of oxy- and deoxy-hemoglobin.

22. The method of claim 21, wherein calculating a tissue oxygenation value based on fitting the tissue spectrum in the visible wavelength range to the absorption spectra of oxy- and deoxy-hemoglobin comprises estimating a blood volume fraction and an oxygen saturation of hemoglobin HbO₂/(Hb+HbO₂) in mixed arterio-venous vasculature.

23. The method of claim 21, further comprising inserting the probe adjacent to the tissue.

24. The method of claim 23, wherein inserting the probe comprises performing laparoscopic surgery on a subject.

25. The method of claim 23, wherein inserting the probe comprises performing cosmetic surgery on a subject.

26. The method of claim 19, wherein the tissue comprises an anastomosis, a repositioned flap in cosmetic surgery, or an effected distal region in vascular surgery.

27. The method of claim 21, wherein the wavelength of the light is from about 480 to about 700 nm.

28. The method of claim 21, wherein the light received by the second fiberoptic fiber comprises a diffuse reflectance spectrum.
